(19) United States
(12) Patent Application Publication
(10) Pub. No.: US 2010/0319696 A1
(43) Pub. Date: Dec. 23, 2010

(54) EJECTION LIQUID AND EJECTION METHOD

(75) Inventors: Hideki Kaneko, Yokohama-shi (JP); Masaru Sugita, Tokyo (JP); Yohei Masada, Kawasaki-shi (JP); Naoko Sakurada, Yokohama-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/526,369

(22) PCT Filed: Jan. 31, 2008
(86) PCT No.: PCT/JP2008/051987

§ 371 Date: Aug. 7, 2009

(30) Foreign Application Priority Data
Feb. 16, 2007 (JP) 2007-036602

(51) Int. Cl. A61M 15/00

(52) U.S. Cl. 128/203.27

(57) ABSTRACT

To stably eject a solution containing at least one type of insulin, by the inkjet method, the present invention provides an ejection liquid characterized by having at least one compound selected from a non-amino acid polycarboxylic acid (excluding citric acid), a non-amino acid nitrogen-containing polycarboxylic acid, and salts thereof, added to a solution containing at least one type of insulin.

### TECHNICAL FIELD

The present invention relates to a liquid composition that contains at least one type of insulin and is suitable for ejection, and a method of its ejection.

### BACKGROUND ART

Many attempts are currently underway to use protein solutions in the form of liquid droplets. Examples are the application of liquid droplet forming technology to protein solutions for transmucosal administration as a method of drug delivery, and for making biochips and biosensors that need very small amounts of protein. In addition, the use of fine droplets of protein is also drawing attention for the control of protein crystallization and in the screening of physiologically active substances (see Japanese Patent Application Laid-Open No. 2002-355025; Allain L R et. al. “Fresenius J. Anal. Chem.”, Vol. 371, p. 146-150, 2001; and Howard E I, Cachau R E, “Biotechniques”, Vol. 33, p. 1302-1306, 2002).

In recent years, proteins, especially enzymes and useful proteins with physiological activity can be mass-produced, by recombinant gene technology. Therefore, creating liquid droplets of protein can become a useful method for the exploration and utilization of novel drugs, and also in applied fields. Among these, the method of using microdroplets for the administration of various types of drugs to patients is gaining importance. In particular, the method of administering proteins, peptides, and other biological materials through the lungs has become important. The lungs have the large alveolar surface area of 50 to 140 m²; the epithelium, which is an absorption barrier, has the very small thickness of 0.1 μm; and enzyme activity is also less than in the gastrointestinal tract. Because of this, transpulmonary administration has been drawing attention as an alternative route to injection for delivering macromolecular peptide drugs, of which insulin is a typical example.

Insulin has been drawing special attention among macromolecular peptide drugs that can be administered through the lungs. Patients with Type 1 diabetes cannot produce insulin in their bodies, and therefore, insulin has to be administered to them before meals. Examples of insulin include ordinary insulin, fast-acting insulins like insulin aspart and insulin lispro, and sustained release insulins like insulin glargine and insulin detemir. Administering an insulin injection before each meal is painful and involves the risk of infection, and therefore, administration of insulin through the pulmonary route, which does not have these drawbacks, is drawing attention.

Generally, intrapulmonary deposition of drug microdroplets is known to be largely dependent on the aerodynamic particle size thereof. Droplets 1 to 5 μm in size with a narrow particle size distribution need to be administered with high reproducibility, especially for delivery to alveoli deep inside the lungs.

Use of the liquid ejection principle used in inkjet printing for obtaining liquid droplets of drug solution with a narrow particle size distribution has been reported (see specification of U.S. Pat. No. 5,894,841 and Japanese Patent Application Laid-Open No. 2002-248171). In liquid ejection by the inkjet method, the liquid to be ejected is led to a small chamber and physical force is applied on the liquid, thereby ejecting droplets from orifices. The method of ejection can be any one of those known in the art, which include generating air bubbles with the help of an electrothermal transducer such as a thin film resistor and discharging the droplets through nozzles (thermal inkjet method), and discharging the liquid directly from the orifices on the chamber with the help of an electromechanical transducer such as a piezo oscillator (piezo inkjet method).

A problem associated with creating droplets of insulin solutions using the principle of the inkjet method is that the physical force (pressure or shear force for example) that comes into play at the time of ejection, or the high surface energy that is peculiar to microdroplets, destabilizes the structure of the insulin. When using the thermal inkjet method, the drug liquid is subjected to thermal energy also. The tertiary structure of insulin is rather fragile, and when this structure is destroyed, it leads to aggregation and decomposition of insulin, which sometimes affect normal ejection. The aforesaid physical effects are far larger than the shear stress or thermal energy involved in ordinary stirring or heat treatment. (For instance, in the thermal inkjet method, a load of about 300° C. and a pressure of 90 atmospheres is applied momentarily). More than one type of physical force is also simultaneously applied. Because of this, the stability of insulin is very prone to decrease, in comparison to the normal situations under which insulin is handled. Once this problem arises, the insulin gets aggregated at the time of droplet formation, causing nozzle blockage and making it difficult to eject droplets.

Droplets of size 1 to 5 μm, which is a suitable size for pulmonary inhalation, are smaller than the droplets of printers commercially available at present. Therefore, the droplets experience even greater surface energy and shear force. This makes it very difficult to eject insulin in the form of fine droplets suitable for pulmonary inhalation.

Thus, it is essential to develop ejection liquids that can be used for ejecting insulin in a stable manner.

The addition of surfactants, glycerol, various saccharides, water-soluble polymers like polyethylene glycol, albumin, etc is a known method of stabilizing insulin. But the addition of these compounds has little or no effect on improving the stability of ejection of insulin by the thermal inkjet method.

Moreover, the addition of additives like polyols such as ethylene glycol and glycerin, and humectants like urea, suited for inks used in inkjet printing, has little effect on improving the ejection performance of insulin solutions.

As for the composition of insulin liquids suitable for forming droplets by the thermal inkjet method, a composition containing a surface tension controlling compound and a humectant has been disclosed (see pamphlet of International Publication No. WO 02/094342). In this WO method, a surfactant or a water-soluble polymer, such as polyethylene glycol, is added for improving the stability of the protein or peptides, typically insulin, in the solution that is made into droplets, by adjusting the surface tension and viscosity, and by providing moisturizing action.

International Publication No. WO 02/094342, however, provides no detailed description about the stability of the ejection. In an examination conducted by the present inventors, the addition of surfactants and water-soluble polymers did not show sufficient effect when the insulin concentration in the liquid was high. Besides, the majority of the surfactants had no effect, and it became clear that the ejection stability of insulin solutions is not determined by the surface tension, viscosity, or the moisturizing action.

As discussed above, known insulin solutions of the type described above do not have satisfactory stability of ejection in the inkjet method.

### DISCLOSURE OF THE INVENTION

The present invention is based on the discovery of a composition having higher ejection stability than the hitherto known ejection liquid. In short, the purpose of the present invention is to provide an ejection liquid (liquid composition) for stable ejection of solutions containing insulin by the inkjet method, and a method of ejection suitable for such an ejection liquid.

The ejection liquid of the present invention is characterized by containing insulin, a non-amino acid polycarboxylic acid (excluding citric acid) represented by General Formula (1) given below or a salt thereof, and a liquid medium, and is meant to be used for ejection by imparting energy through electrothermal or electromechanical transducers.

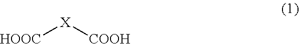

In Formula (1), X represents an optionally branched alkyl group with 1 carbon atom or more and 12 carbon atoms or less, and the main chain optionally has a heteroatom(s). Furthermore, the main chain and the branched side chains optionally have one or more hydroxyl groups or carboxyl groups.

The liquid ejection cartridge of the present invention is characterized by having a reservoir in which the aforesaid ejection liquid is stored, and an ejection head having electrothermal transducers that impart thermal energy or electromechanical transducers that impart mechanical energy to the ejection liquid.

The inhaler of the present invention is characterized by having the aforesaid cartridge, and a suction port for inhaling the liquid ejected from the liquid ejection portion of the ejection head of the cartridge.

The ejection method of the present invention is characterized in that a liquid composition that contains insulin, a non-amino acid polycarboxylic acid (excluding citric acid) represented by General Formula (1) given above or a salt thereof, and a liquid medium, is ejected by providing energy from electrothermal transducers or electromechanical transducers.

According to the present invention, by adding the specified type of polycarboxylic acid to the insulin-containing solution, ejection liquids that can be stably ejected by the inkjet method can be obtained. This ejection liquid is ejected through a portable ejection device. The insulin is delivered to the lungs through inhalation of the ejected droplets, and gets absorbed into the body. Furthermore, the ejection liquid can be used to prepare biochips and biosensors by ejecting it on substrates using the method described above.

Other features and advantages of the present invention will be apparent from the following description taken in conjunction with the accompanying drawings, in which like reference characters designate the same or similar parts throughout the figures thereof.

### BRIEF DESCRIPTION OF THE DRAWING

FIG. 1 is a schematic diagram of the liquid ejection cartridge unit used in inhalers;

FIG. 2 is an oblique perspective view of an inhaler according to the present invention; and

FIG. 3 is an oblique perspective view of the inhaler of FIG. 2 with an opened access cover.

### BEST MODES FOR CARRYING OUT THE INVENTION

Preferred embodiments of the present invention will now be described in detail in accordance with the accompanying drawings.

The term “insulin” used in the present invention means insulin as well as any polypeptide produced by partial modification of the amino acid sequence of insulin, and is soluble or dispersible in an aqueous solution. The insulin can be a chemically synthesized one, or a recombinant form of purified natural insulin. By chemically modifying the insulin by covalent bonding to amino acid residues of the insulin, its effectiveness, such as by prolonging its therapeutic effect, can be improved.

Various types of insulin, for which ejection is desirable, are used to carry out the present invention. The type of insulin used in the present invention is not limited; any type can be used as long as it has physiological activity on the living body, and retains the activity in the living body. Most typically, droplet formation of insulin according to the present invention can be suitably applied for delivering therapeutically useful insulin to the lungs.

Examples of insulin used here include insulin; and insulins with modified amino acid sequences, such as insulin aspart, insulin lispro, insulin glargine, and insulin detemir; etc. The peptide parts of insulin having the whole or part of the main structure of the aforesaid substances, and having at least a part of the biological properties of insulin, may also be used. Those containing the aforesaid substances modified with water-soluble polymers like PEG and PVA may also be used. That protein or peptide that has been modified with PEG, PVA, etc can be delivered to the lungs, has been demonstrated in Critical Reviews in Therapeutic Drug Carrier Systems, 12 (2&3), 1995.

Furthermore, when preparing biochips or biosensors, the aforesaid substances, which have been modified with reagents like 4-azidobenzoic acid N-hydroxysuccinimide ester for fixing the insulin, may be used.

The content of insulin, at least one type selected from among the different types, in the ejection liquid is decided based on its type and the purpose of its use. Preferably, it is chosen from the range of 1 μg/ml to 200 mg/ml, more preferably from 0.1 mg/ml to 60 mg/ml.

In ejecting the ejection liquid of the present invention by imparting thermal energy, improved ejection performance can be seen most prominently when the thermal energy is provided by electrothermal transducers and the thermal inkjet method is used. Therefore, in the description hereinafter, we shall mainly discuss the configuration based on the principle of the thermal inkjet method. However, the piezo inkjet method, in which the liquid is ejected through nozzles with the help of oscillating pressure of electromechanical transducers (piezo elements, for instance) that impart mechanical energy to the ejection liquid, may also be used in the present invention. The method of ejection can be selected according to the type of insulin to be ejected. In the present invention, among the inkjet technologies normally used in printers, the inkjet method in which thermal energy is provided with electrothermal transducers is referred to as the “thermal inkjet method”, and the method in which mechanical energy is provided through electromechanical transducers is referred to as “the piezo inkjet method”. These terms are used in relation to the insulin solution. However, this only means that the ejection energy is imparted to the solution according to the principle of the specified inkjet method.

When using the thermal inkjet method, the accuracy and reproducibility of the ejection orifice diameter, the amount of heat in the heat pulse used for the ejection, the size of the microheaters used, etc. can be improved for each liquid ejection unit. Therefore, a narrow droplet size distribution can be achieved. Apart from this, as the production cost of the head is low, it has high applicability for a small-sized device where the head needs to be changed often. Thus, an ejection device using the thermal inkjet method is particularly suitable if portability and user-friendliness are required in the device.

The addition of a surfactant, or a solvent like ethylene glycol, is a generally known practice for improving the ejectability of ink in the inkjet method. However, the addition of these substances alone was found to be not sufficient for improving the ejectability of solutions containing at least one type of insulin, and therefore, new additives were required.

According to the investigations conducted by the present inventors, when insulin at a concentration that showed effective physiological activity was ejected using the thermal inkjet method without adding an additive, the ejection almost stopped at the ejection frequency of 10 kHz or higher.

As a result of painstaking investigations, the present inventors discovered that a solution containing at least one type of insulin as the active ingredient can be stably ejected using the principle of the inkjet method if a non-amino acid polycarboxylic acid (excluding citric acid) is added to the solution.

The non-amino acid polycarboxylic acid may be added in the form of a salt, and salts of alkali metals such as sodium or potassium, or the ammonium salt, etc may be used.

The reason for the addition of the aforesaid polycarboxylic acid and the like making a significant contribution to the ejection stability of the insulin solution is not clearly understood, but it is believed to be as follows. The aforesaid types of polycarboxylic acids facilitate the dissolving of insulin at high concentrations even in insulin solutions with a pH close to its isoelectric point. The carboxyl group is believed to contribute to the improved solubility. Therefore, when insulin becomes insoluble because of load during ejection, the aforesaid type of carboxylic acid promptly redissolves it because of its high dissolving power. Here, “load during ejection” means mainly heat in the thermal inkjet method and mainly pressure in the piezo inkjet method. In both these methods, a large load momentarily falls on the solution at the time of ejection. In other words, the aforesaid type of polycarboxylic acid promptly redissolves, in the water, the small amounts of insoluble insulin invariably generated. This, in turn, is believed to stabilize the transfer of thermal energy to the solution and make the ejection stable.

The effect of improved ejectability of the ejection liquid of the present invention becomes even more prominent especially with heads that eject liquid using thermal energy, operated at a high frequency.

The compound represented by the General Formula (I) shown below is preferable as the non-amino acid polycarboxylic acid used in the present invention.

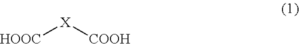

In Formula (1), X represents an optionally branched alkyl group with 1 carbon atom or more and 12 carbon atoms or less, and the main chain optionally has a heteroatom(s). Furthermore, the main chain and the branched side chains optionally have one or more hydroxyl groups or carboxyl groups. The hydrogen atoms of the alkyl group at X may be optionally substituted by halogen atoms or hydroxyl groups at one or more sites. Furthermore, salts of these compounds also may be used. A polymer having a repeating unit of such compound may also be used. It may also be a surfactant that contains such a compound within its structure.

The molecular weight of the non-amino acid polycarboxylic acid is preferably 104 to 2000, and more preferably 104 to 500. A compound with solubility greater than 0.1% by weight in water in the neutral range (pH 5.5 to 8.5) can be used for achieving the desired effect.

The polycarboxylic acids to be added to the insulin solution in the present invention do not include compounds that are simultaneously amino acids and also polycarboxylic acids, such as glutamic acid.

The non-amino acid polycarboxylic acids of the present invention include polycarboxylic acids that contain an N atom(s) in the main chain of X in the General Formula (1) given above.

Among the non-amino acid polycarboxylic acids represented by General Formula (1), compounds with n/m=0.4 or more, or more preferably 0.5 or more, where m is the total number of carbon atoms and heteroatoms in the main chain of the alkyl group X and n is the number of carboxyl groups, are preferable. The n/m ratio is a parameter that reflects the proportion of the additive molecule occupied by carboxyl groups. Carboxyl groups are believed to contribute to improving the solubility of insulin. Therefore, it is believed that the greater the value of this parameter, the higher is the probability of the carboxyl groups acting on the insulin, improving the solubility of the insulin, and stabilizing the ejection of the solution.

Examples of preferred polycarboxylic acids to be used in the present invention include malonic acid, succinic acid, glutaric acid, 1,2,3-propanetricarboxylic acid, adipic acid, malic acid, tartaric acid, ethylenediamine tetraacetic acid (EDTA), diethylenetriamine pentaacetic acid (DTPA), and salts thereof.

0.1 to 50 parts by weight of the non-amino acid polycarboxylic acid can be added for 1 part by weight of insulin. More preferably, 0.25 to 25 parts by weight, and most preferably, 0.5 to 10 parts by weight of the non-amino acid polycarboxylic acid is added, for 1 part by weight of insulin.

The content of the non-amino acid polycarboxylic acid in the ejection liquid is to be selected, depending on the type and content of insulin in the liquid, and it is selected preferably in the range of 1 μg/ml to 2.0 g/ml, more preferably 10 μg/ml to 200 mg/ml.

The composition of the liquid medium, from the point of view of solubility of insulin, can be water or a mixed liquid medium mainly consisting of water and containing water-soluble organic solvents like alcohol. Specific examples of water-soluble organic solvents are amides like dimethylformamide, and dimethylacetamide; ketones like acetone; ethers like tetrahydrofuran and dioxane; polyalkylene glycols like polyethylene glycol and polypropylene glycol; alkylene glycols with the alkylene group having 2 to 5 carbon atoms, like ethanol, ethylene glycol, propylene glycol, butylene glycol, triethylene glycol, 1,2,6-hexanetriol, thiodiglycol, hexylene glycol, and diethylene glycol; glycerin; lower alkyl ethers of polyhydric alcohols, like ethylene glycol monomethyl (or ethyl)ether, diethylene glycol monomethyl (or ethyl)ether, triethylene glycol monomethyl (or ethyl)ether; and N-methyl-2-pyrrolidone.

The content of the aforesaid water-soluble organic solvent can be generally 0.1 to 40% by weight, more preferably 1 to 30% by weight, with respect to the total weight of the ejection liquid. The content of water in the liquid medium is in the range of 30 to 95% by weight. Less then 30% by weight of water is not suitable, as it makes the solubility, etc of the protein poor, and the viscosity of the ejection liquid too high. On the other hand, with more than 95% by weight of water, the volatile components become too much and sufficient fixing property cannot be obtained.

In the present invention, the insulin, the polycarboxylic acid, etc may be either premixed or mixed just before ejection. But they can be in a uniformly mixed condition by the time of ejection.

In the embodiments of the present invention, a surfactant can be used to improve the efficiency of insulin absorption in the lungs. There is no restriction on the surfactant that can be used. Typical examples of such surfactants include sorbitan fatty acid esters such as sorbitan monocaprilate, sorbitan monolaurate, and sorbitan monopalmitate; N-acyl amino acids, which are surfactants having an amino acid as a hydrophilic group, such as N-coconut oil fatty acid glycine, N-coconut oil fatty acid glutamic acid, and N-lauroyl glutamic acid; fatty acid salts of an amino acid; glycerin fatty acid esters such as glycerin monocaprylate, glycerin monomyristate, and glycerin monostearate; polyglycerin fatty acid esters such as decaglyceryl monostearate, decaglyceryl distearate, and decaglyceryl monolinolate; polyoxyethylene sorbitan fatty acid esters such as polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, and polyoxyethylene sorbitan tristearate; polyoxyethylene sorbitol fatty acid esters such as polyoxyethylene sorbitol tetrastearate and polyoxyethylene sorbitol tetraoleate; polyoxyethylene glycerin fatty acid esters such as polyoxyethylene glyceryl monostearate; polyethylene glycol fatty acid esters such as polyethylene glycol distearate; polyoxyethylene alkyl ethers such as polyoxyethylene lauryl ether; polyoxyethylene polyoxypropylene alkyl ethers such as polyoxyethylene polyoxypropylene glycol ether, polyoxyethylene polyoxypropylene propyl ether, and polyoxyethylene polyoxypropylene cetyl ether; polyoxyethylene alkylphenyl ethers such as polyoxyethylene nonylphenyl ether; polyoxyethylene hardened castor oils such as polyoxyethylene castor oil and polyoxyethylene hardened castor oil (polyoxyethylene hydrogenated castor oil); polyoxyethylene beeswax derivatives such as polyoxyethylene sorbitol beeswax; polyoxyethylene lanolin derivatives such as polyoxyethylene lanolin; polyoxyethylene fatty acid amides and the like, having HLB value of 6 to 18, such as polyoxyethylene stearic acid amide; anionic surfactants like alkyl sulfates with the alkyl group having 8 to 18 carbon atoms, such as sodium cetyl sulfate, sodium lauryl sulfate, and sodium oleyl sulfate; polyoxyethylene alkyl ether sulfates where the average number of added moles of ethylene oxide is 2 to 4 and the alkyl group has 8 to 18 carbon atoms, such as sodium polyoxyethylene lauryl sulfate; alkyl benzene sulfonates having 8 to 18 carbon atoms in the alkyl group, such as sodium lauryl benzene sulfonate; alkyl sulfosuccinate ester salts having 8 to 18 carbon atoms in the alkyl group, such as sodium lauryl sulfosuccinate; a natural surfactant such as lecithin and glycerophospholipid; sphingophospholipids such as sphingomyelin; and sucrose fatty acid esters of a fatty acid having 8 to 18 carbon atoms. These surfactants can be added singly or in combinations of two or more to the ejection liquid (liquid composition) of the present invention.

In embodiments of the present invention, antimicrobial agents, germicidal agents, and preservatives may be added, to eliminate the effects of microorganisms. Examples of such agents include quaternary ammonium salts such as benzalkonium chloride and benzatonium chloride; phenol derivatives such as phenol, cresol, and anisole; benzoic acids such as benzoic acid and paraoxybenzoic acid ester; and sorbic acid.

In embodiments of the present invention, oils, glycerin, ethanol, urea, cellulose, polyethylene glycol, and alginates may be added to increase physical stability of the ejection liquid during storage. In addition, to enhance the chemical stability, ascorbic acid, cyclodextrin, tocopherol, or any other antioxidant may be added.

A pH regulator or buffer may be added to adjust the pH of the ejection liquid. Examples include ascorbic acid, dilute hydrochloric acid, and dilute sodium hydroxide, and also buffers such as of sodium hydrogen phosphate, sodium dihydrogen phosphate, potassium hydrogen phosphate, potassium dihydrogen phosphate, PBS, HEPES, and Tris.

Aminoethylsulfonic acid, potassium chloride, sodium chloride, glycerin, or sodium hydrogen carbonate may be added to the liquid as an isotonizing agent.

When using the ejection liquid of the present invention as a spray liquid, saccharides such as glucose and sorbitol, sweeteners such as aspartame, menthol, and various flavors may be added as flavoring agents. Apart from the hydrophilic ones, hydrophobic compounds such as those in the form of oil can also be used.

Also, if necessary, various additives suited for the purpose for which the ejection liquid is to be used, such as surface conditioners, viscosity regulators, solvents, and humectants, may be added in suitable amounts. Specific examples of such additives that can be added include hydrophilic binders, hydrophobic binders, hydrophilic thickeners, hydrophobic thickeners, glycol derivatives, alcohols, and electrolytes. These may be added singly or in combinations. Among the various examples of aforesaid typical additives, it is preferable to use those meant for use in medicines, and listed as permissible subsidiary components for therapeutic liquid formulations in a pharmacopoeia in preparing a drug liquid for treatment, or those permitted for use in foods or cosmetics.

The extent to which the various substances described above are added as additives depends on the type of insulin used, but generally it is preferable to keep it within the range of 0.001 to 40% by weight, more preferably 0.01 to 20% by weight. Besides this, the amount of the aforesaid additives also depends on their type, quantity and combination. However, from the viewpoint of ejectability, it is preferable to keep it in the range of 0.01 to 100 parts by weight for one part by weight of insulin.

When ejecting the insulin solution by the thermal inkjet method, it is preferable to operate the head at a driving frequency that is as low as possible. “Driving frequency” in the present invention means the number of ejection pulses applied per second to an electrothermal transducer in the case of the thermal inkjet method. The reason for the stability of ejection differing under different driving frequencies is believed to be that when the ejection liquid is heated by the electrothermal transducer of the thermal inkjet head, a part of the insulin becomes insoluble in water and prevents the transmission of energy from the electrothermal transducer to the solution. It is believed that with a low driving frequency, even if insoluble matter is generated temporarily, it gets redissolved by the time of the next driving pulse, whereas at high driving frequencies, the solubility does not recover sufficiently and the ejection stability declines as a result. The ejection has, however, to be done at a frequency higher than a certain value when a large amount of solution is to be efficiently ejected. When the insulin solution is ejected by the thermal inkjet method without adding any additives, the ejection almost stops at the ejection frequency of 10 kHz or higher. But, with the present invention, stable ejection can be achieved at ejection frequencies of 10 kHz or higher. The driving frequency suited for the ejection liquid of the present invention is 0.1 kHz or higher and 100 kHz or lower, more preferably 1 kHz or higher and 30 kHz or lower.

When using the ejection liquid of the present invention for making biochips or biosensors, a system that is substantially the same as that of current commercially available inkjet printers can be used.

On the other hand, the liquid ejection device of the present invention has a thermal inkjet head that can eject fine droplets of the ejection liquid by the thermal inkjet method. The electrothermal transducers of individual nozzles that constitute the head part can be configured in such a way that they can be driven independently form each other. In that case, an electrical connection portion that provides the connections for a plurality of control signals, etc needed for independently driving each electrothermal transducer, is integrated with wires that lead to individual electrothermal transducers. In addition, the liquid ejection cartridge can be structured in such a way that the reservoir of storing the ejection liquid therein and the ejection head, which has the electrothermal transducers for imparting thermal energy to the ejection liquid, are integrated.

Next, we shall discuss the use of the ejection liquid of the present invention for atomization, especially in inhalers. The inhaler preferably has a structure where the part in which the ejection liquid is turned into fine droplets is independent of the part where the atomized fine droplets are mixed with a carrier airflow. This separation of the part that converts the liquid into fine droplets and the part where the droplets are mixed with the carrier airflow enables uniform adjustment of the amount of ejection. In other words, when a person inhales the airflow, the amount of protein or peptide active ingredient, i.e., the predetermined dose for single administration, can be adjusted more uniformly. Also, as mentioned above, if the ejection head portion is configured with a plurality of ejection units, each unit to eject a different active ingredient through a number of ejection orifices provided, the amounts of more than one active ingredient ejected can also be controlled independently.

Besides this, if an ejection head based on the thermal inkjet method, which enables a high density arrangement of ejection orifices per unit area, is used as the atomizing mechanism, it becomes easy to miniaturize the inhaler to enable users to carry it with them.

In inhalers for pulmonary inhalation, it is important for the size of the droplets contained in the airflow to be 1 to 5 μm and the particle size range to be narrow. Moreover, for the inhaler to be used as a portable device it needs to be compact.

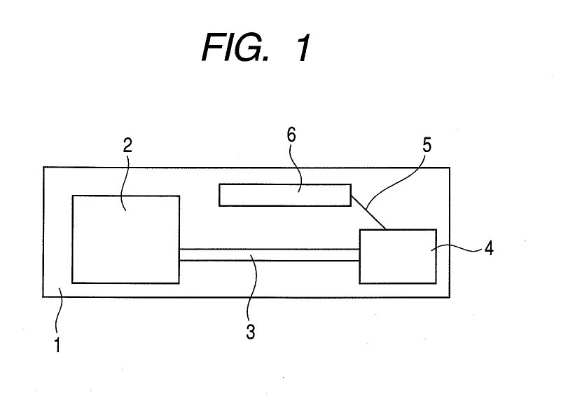

An example of a liquid ejection cartridge of the aforesaid type of inhaler is illustrated schematically in FIG. 1. In this liquid ejection cartridge, a casing 1 contains an ejection head part 4, a reservoir 2 for storing the ejection liquid, a liquid passage 3 for supplying the liquid form the reservoir 2 to the head part 4, and an electrical connection portion 6 for exchanging driving signals, control signals, etc with a controller that controls the driving of each ejection unit of the head part 4; where a head cartridge unit has a structure in which an inner wire 5 between the head part 4 and the electrical connection portion 6 is formed integrally. The head cartridge unit has a configuration that permits its detachment from the inhaler when required. As the head part 4, a liquid ejection head with the structure described in Japanese Patent Application Laid-Open No. 2003-154665 is suitable.

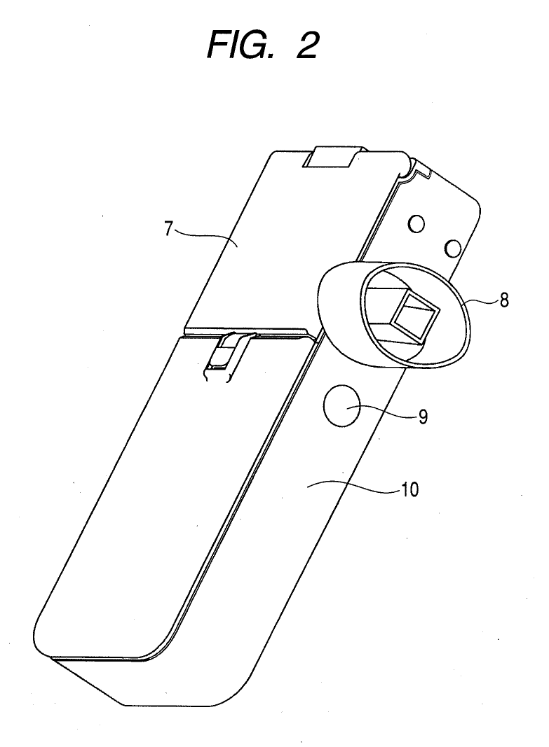

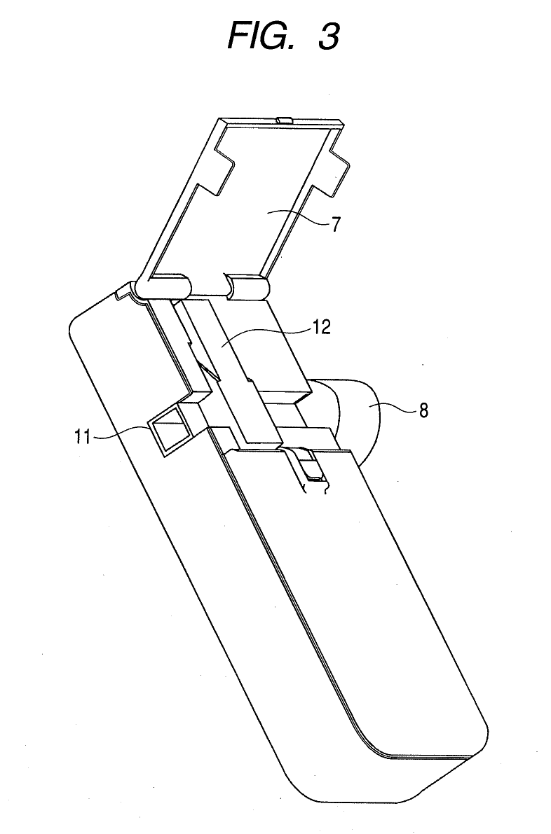

An example of a portable inhaler having a head cartridge unit with the aforesaid structure is described below referring to FIGS. 2 and 3. The inhaler illustrated in FIGS. 2 and 3 has an exemplary miniaturized configuration so that users can carry it with them at all times for medical purpose.

FIG. 2 is an oblique perspective view of the inhaler. In this inhaler, an inhaler body 10 and an access cover 7 together form a housing. A controller, a power source (battery), etc (not shown) are placed in the housing. FIG. 3 illustrates the condition where the access cover 7 is opened. A head cartridge unit 12 becomes visible when the access cover 7 is opened. When the user sucks on the inhaler, air is drawn into an inhaler through an air inlet 11, and is led to a mouthpiece 8 (suction port) where it is mixed with the liquid droplets ejected from the ejection orifices provided on the head part 4 of the head cartridge unit 12, to form a mixed airflow. This mixed airflow then proceeds to a mouthpiece outlet having a shape that the user can hold in his or her mouth. When the user inserts the tip of the mouthpiece 8 into the mouth, holds it by the teeth, and breathes in, the liquid droplets ejected from a liquid ejection part are led to the mouthpiece 8, and can be effectively inhaled.

The head cartridge unit 12 may be so configured that it is detachable, as needed, from the inhaler.

By adopting the configuration shown in FIGS. 2 and 3, the fine droplets formed can be delivered naturally, along with the inhaled air, to the throat and into the tracheae of the user. Therefore, the amount of liquid atomized, i.e., the dose of the active ingredient, does not depend on the volume of the air inhaled, and can be controlled independently. Examples 1 to 11 and Comparative Examples 1 to 8

The present invention will be described in below greater detail, referring to some examples. However, the invention is in no way restricted by these examples. Here, “%” means % by weight.

The procedure used for preparing the insulin solution is described below. Firstly, a suitable concentration of insulin (manufactured by Sigma-Aldrich Co.) was dissolved in 0.01 M aqueous hydrochloric acid. While stirring this solution, a 50 mg/ml aqueous solution of a certain polycarboxylic acid, the pH of which had been adjusted to 7 with 2 M NaOH, was added to obtain a predetermined concentration. After that, the pH of the solution was adjusted to 7 using 0.1 M NaOH, and water was added to make up the volume and achieve a predetermined insulin concentration.

For preparing insulin aspart and insulin lispro solutions, a 50 mg/ml aqueous solution of a certain polycarboxylic acid, the pH of which had been adjusted to 7.4 with 2 M NaOH, was added, while stirring, to NovoRapid (manufactured by Novo Nordisk Co,) or Humalog (manufactured by Eli Lilly and Co.) to obtain a predetermined concentration. After that, water was added to make up the volume and achieve a predetermined insulin aspart or insulin lispro concentration.

Each ejection liquid prepared by the procedure described above was filled in a head having the same configuration as in an inkjet printer (trade name: PIXUS950i, manufactured by Canon Inc.) and the ejection head was driven by the ejection controller. The ejectability at the frequency of 24 kHz was evaluated. It was graded as “A” if ejection continued for 10 minutes, and as “C” if it stopped before that. In the Comparative Examples, the ejection liquids were prepared by adding aqueous solutions of compounds other than the specified polycarboxylic acids to the insulin solutions, and ejection tests were carried out as in the examples. Table 1 below gives the compositions examined in the examples and Comparative Examples and the ejectability evaluated. The “unit” of insulin concentration used in Table 1 is a standard dose unit commonly used internationally for insulins.

TABLE 1

| | | | | | Concentration | | |
|---|---|---|---|---|---|---|---|
| | | | | Additive for | of additive | | |
| | Insulin | Insulin | | improving | for improving | | |
| | type | concentration | | ejectability | ejectability | | Ejectability |
| Example 1 | Insulin | 2.0 | mg/mL | Tartaric acid | 10.0 | mg/mL | A |
| Example 2 | Insulin | 2.0 | mg/mL | Succinic acid | 1.0 | mg/mL | A |
| Example 3 | Insulin | 2.0 | mg/mL | Glutaric acid | 1.0 | mg/mL | A |
| Example 4 | Insulin | 2.0 | mg/mL | 1,2,3-propane | 1.0 | mg/mL | A |
| | | | | tricarboxylic | | | |
| | | | | acid | | | |
| Example 5 | Insulin | 2.0 | mg/mL | EDTA | 1.0 | mg/mL | A |
| Example 6 | Insulin | 2.0 | mg/mL | DTPA | 1.0 | mg/mL | A |
| Example 7 | Insulin | 10.0 | mg/mL | EDTA | 10.0 | mg/mL | A |
| Example 8 | Insulin | 80 | units/mL | EDTA | 1.0 | mg/mL | A |
| | aspart | | | | | | |
| Example 9 | Insulin | 80 | units/mL | DTPA | 1.0 | mg/mL | A |
| | aspart | | | | | | |
| Example 10 | Insulin | 80 | units/mL | EDTA | 1.0 | mg/mL | A |
| | lispro | | | | | | |
| Example 11 | Insulin | 80 | units/mL | DTPA | 1.0 | mg/mL | A |
| | lispro | | | | | | |
| Comparative | Insulin | 2.0 | mg/mL | Nil | — | | C |
| Example 1 | | | | | | | |
| Comparative | Insulin | 2.0 | mg/mL | Tween80 | 1.0 | mg/mL | C |
| Example 2 | | | | | | | |
| Comparative | Insulin | 2.0 | mg/mL | Glycerin | 10.0 | mg/mL | C |
| Example 3 | | | | | | | |
| Comparative | Insulin | 10.0 | mg/mL | PEG4000 + | 60.0 mg/mL + | | C |
| Example 4 | | | | Tween80 | 1.0 mg/mL | | |
| Comparative | Insulin | 80 | units/mL | Nil | — | | C |
| Example 5 | aspart | | | | | | |
| Comparative | Insulin | 80 | units/mL | Tween80 | 1.0 | mg/mL | C |
| Example 6 | aspart | | | | | | |
| Comparative | Insulin | 80 | units/mL | Nil | — | | C |
| Example 7 | lispro | | | | | | |
| Comparative | Insulin | 80 | units/mL | Tween80 | 1.0 | mg/mL | C |
| Example 8 | lispro | | | | | | |

The ejection liquids of Examples 1 to 11 were analyzed by HPLC (Measurement conditions: analyzer: JASCO Corporation; column: YMC-Pack Diol-200 500×8.0 mm ID; eluent 0.1 M KH₂PO₄—K₂HPO₄ (pH 7.0) containing 0.2 M NaCl; flow rate: 0.7 ml/min; temperature: 25° C.; detection: UV at 215 nm) before and after the ejection, to check for changes in the composition of each ejection liquid.

The results of HPLC analysis showed no change in peak position or peak area, and in the liquid composition, between before and after the ejection in Examples 1 to 11.

The n/m ratio of the polycarboxylic acids used in Examples 1 to 11 are given in Table 2. Here, m is the total number of carbon atoms and heteroatoms in the main chain of the alkyl group X, and n is the number of carboxyl groups.

TABLE 2

| Compound | Structure | m | n | n/m |
|---|---|---|---|---|
| Succinic acid | 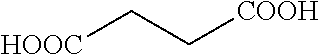 | 2 | 2 | 1 |
| Glutaric acid | 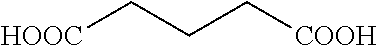 | 3 | 2 | 0.67 |
| Tartaric acid | 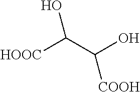 | 2 | 2 | 1 |
| 1,2,3-propane tricarboxylic acid | 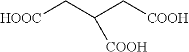 | 3 | 3 | 1 |
| EDTA | 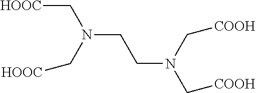 | 6 | 4 | 0.67 |
| DTPA | 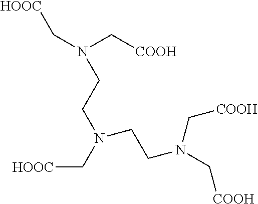 | 9 | 5 | 0.56 |

### Examples 12 to 19

Ejection tests were conducted at different driving frequencies after adding tartaric acid, glutaric acid, 1,2,3-propane tricarboxylic acid or EDTA, among the polycarboxylic acids used in the earlier examples. The test conditions and test procedures other than the driving frequency were the same as in Examples 1, 3, 4 and 5. The driving frequencies used and the ejectability evaluated are given in Table 3 below.

TABLE 3

| | | Ejection | | |
|---|---|---|---|---|
| | Additive | frequency (Hz) | | Ejectability |
| Example 12 | Tartaric acid | 1000 | Hz | A |
| Example 13 | Tartaric acid | 15000 | Hz | A |
| Example 14 | Glutaric acid | 1000 | Hz | A |
| Example 15 | Glutaric acid | 15000 | Hz | A |
| Example 16 | 1,2,3-propane | 1000 | Hz | A |
| | tricarboxylic acid | | | |
| Example 17 | 1,2,3-propane | 15000 | Hz | A |
| | tricarboxylic acid | | | |
| Example 18 | EDTA | 1000 | Hz | A |
| Example 19 | EDTA | 15000 | Hz | A |

As described above, ejection stability was maintained even when different ejection frequencies were used. The results of HPLC analysis showed no change in peak position or peak area, and in the liquid composition, between before and after the ejection in these examples also.

### Examples 20 to 30 and Comparative Examples 9 to 16

A liquid ejection head of the thermal inkjet method having 3 μm diameter nozzles was prepared and 30% aqueous ethanol solution was filled in the reservoir connected to the ejection head. The liquid was ejected from the ejection orifices by driving the ejection head using the controller electrically connected to the head. The particle size and size distribution of the droplets obtained were measured and confirmed using a laser diffraction-based particle size distribution analyzer (SprayTech, manufactured by Malvern Instruments), and it was found that the droplets had a sharp particle size distribution around 3 μm.

Each prepared ejection liquid was filled in the reservoir connected to the aforesaid liquid ejection head with 3 μm nozzle diameter. The ejection head was then driven using the ejection controller and the liquid ejected for 1 second (1st round ejection) at frequency 20 kHz and voltage 12 V. After an interval of 60 seconds, the liquid was ejected again for 1 second (2nd round ejection). Up to 50 rounds of this operation were done, and the sustainability of the ejection was checked through visual observation. A liquid that could be ejected 50 times was graded as “A”, a liquid where the droplet ejection stopped between 15 and 50 rounds of ejection was graded as “B”, and a liquid where the ejection stopped in less than 15 rounds was graded as “C”. The ejection liquids were also analyzed with HPLC before and after the ejection test to observe any changes in the composition of each ejection liquid.

For the Comparative Examples, ejection liquids were prepared by adding compounds other than the specified polycarboxylic acids to the insulin solutions, and liquid ejection tests were carried out as in the examples. The compositions of the ejection liquids examined in the examples and Comparative Examples and their evaluated ejectability are given in Table 4.

TABLE 4

| | | | | | Concentration | | |
|---|---|---|---|---|---|---|---|
| | | | | Additive for | of additive | | |
| | Insulin | Insulin | | improving | for improving | | |
| | type | concentration | | ejectability | ejectability | | Ejectability |
| Example 20 | Insulin | 2.0 | mg/mL | Tartaric acid | 10.0 | mg/mL | A |
| Example 21 | Insulin | 2.0 | mg/mL | Succinic acid | 1.0 | mg/mL | A |
| Example 22 | Insulin | 2.0 | mg/mL | Glutaric acid | 1.0 | mg/mL | A |
| Example 23 | Insulin | 2.0 | mg/mL | 1,2,3-propane | 1.0 | mg/mL | A |
| | | | | tricarboxylic | | | |
| | | | | acid | | | |
| Example 24 | Insulin | 2.0 | mg/mL | EDTA | 1.0 | mg/mL | A |
| Example 25 | Insulin | 2.0 | mg/mL | DTPA | 1.0 | mg/mL | A |
| Example 26 | Insulin | 10.0 | mg/mL | EDTA | 10.0 | mg/mL | A |
| Example 27 | Insulin | 80 | units/ml | EDTA | 1.0 | mg/mL | A |
| | aspart | | | | | | |
| Example 28 | Insulin | 80 | units/ml | DTPA | 1.0 | mg/mL | A |
| | aspart | | | | | | |
| Example 29 | Insulin | 80 | units/ml | EDTA | 1.0 | mg/mL | A |
| | lispro | | | | | | |
| Example 30 | Insulin | 80 | units/ml | DTPA | 1.0 | mg/mL | A |
| | lispro | | | | | | |
| Comparative | Insulin | 2.0 | mg/mL | Nil | — | | C |
| Example 9 | | | | | | | |
| Comparative | Insulin | 2.0 | mg/mL | Tween80 | 1.0 | mg/mL | C |
| Example 10 | | | | | | | |
| Comparative | Insulin | 2.0 | mg/mL | Glycerin | 10.0 | mg/mL | C |
| Example 11 | | | | | | | |
| Comparative | Insulin | 10.0 | mg/mL | PEG4000 + | 60.0 mg/mL + | | C |
| Example 12 | | | | Tween80 | 1.0 mg/mL | | |
| Comparative | Insulin | 80 | units/ml | Nil | — | | C |
| Example 13 | aspart | | | | | | |
| Comparative | Insulin | 80 | units/ml | Tween80 | 1.0 | mg/mL | C |
| Example 14 | aspart | | | | | | |
| Comparative | Insulin | 80 | units/ml | Nil | — | | C |
| Example 15 | lispro | | | | | | |
| Comparative | Insulin | 80 | units/ml | Tween80 | 1.0 | mg/mL | C |
| Example 16 | lispro | | | | | | |

In Comparative Examples 9 to 16 no ejection occurred through the thermal inkjet head with nozzle diameter 3 μm. On the other hand, very stable ejection was seen in the examples, confirming the effect of adding the compounds according to the present invention. The results of HPLC analysis showed no change in peak position or peak area, and in the liquid composition, between before and after the ejection in the examples.

These experiments showed that stable ejection of insulin solution is possible at high frequency even under the microdroplet ejection condition of nozzle diameter as small as 3 μm.

The present invention is not limited to the above embodiments and various changes and modifications can be made within the spirit and scope of the present invention. Therefore to apprise the public of the scope of the present invention, the following claims are made.

This application claims the benefit of Japanese Patent Application No. 2007-036602, filed Feb. 16, 2007, which is hereby incorporated by reference herein in its entirety.

1.-6. (canceled)

7. An ejection method comprising imparting energy from electrothermal transducers to a liquid composition containing insulin, a polycarboxylic acid (excluding citric acid) represented by General Formula (1) or ethylenediamine tetraacetic acid (EDTA) or diethylenetriamine pentaacetic acid (DTPA), or a salt thereof, thereby ejecting the liquid composition: 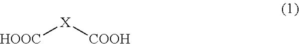
in Formula (1), X represents an optionally branched alkyl group with 1 carbon atom or more and 12 carbon atoms or less, and the main chain and the branched side chains optionally have one or more hydroxyl groups or carboxyl groups.

8. (canceled)

9. The ejection method according to claim 7, wherein the liquid composition is ejected by controlling the driving frequency of the electrothermal transducers at 0.1 kHz or higher and 100 kHz or lower.

10. (canceled)

11. The ejection method according to claim 7, wherein the polycarboxylic acid represented by General Formula (1) is a compound selected from the group comprising succinic acid, glutaric acid, 1,2,3-propane tricarboxylic acid, tartaric acid, and salts thereof.
